(19) United States
(12) Patent Application Publication
(10) Pub. No.: US 2006/0165633 A1
(43) Pub. Date: Jul. 27, 2006

(54) Process for the parmanent reshaping of keratin substances without intermediate rinsing

(75) Inventors: Ly-Lan Nguyen, L'Hay-les-Roses (FR); Anne Sabbach, Rueil-Malmaison (FR)

Correspondence Address: NIXON & VANDERHYE, PC, 901 NORTH GLEBE ROAD, 11TH FLOOR, ARLINGTON, VA, 22203, US

(73) Assignee: L'OREAL

(21) Appl. No.: 11/339,609

(22) Filed: Aug. 12, 2002

Related U.S. Application Data
(63) Continuation of application No. 09/529,175, filed on Apr. 25, 2000, now abandoned.

(30) Foreign Application Priority Data
Oct. 10, 1997 (FR) 97/12713

(51) Int. Cl. A61K 8/73; A61K 8/97

(52) U.S. Cl. 424/70.13; 424/70.2; 424/74

(57) ABSTRACT

The invention concerns a method for the permanent setting of keratin fibres and in particular hair, characterised in that it consists in: applying on the keratin fibres a composition reducing keratin, with adhesive capacity less than 15 g and a viscosity ranging between 0 and 2500 mpa's at a laminar flow rate of 100 s⁻¹ and setting them; after sufficient setting time for reducing keratin, applying an oxidising composition, without intermediate rinsing of the fibres; rinsing after sufficient time for fixing the set fibres and reduced fibres in permanent setting; rinsing with water.

The present invention relates to a novel process for treating keratin substances, and in particular the hair, in order to permanently reshape the substances.

One of the techniques commonly used in cosmetics to imprint a long-lasting shape on the hair consists in reshaping the hair by using a reducing agent and then an oxidizing agent.

The technique most commonly used for permanently reshaping the hair consists, in a first stage, in opening the S—S— disulphide linkages in keratin (cystine) using a composition containing a reducing agent, and then, after the hair thus treated has been rinsed, in reconstituting the said disulphide linkages, in a second stage, by applying to the hair, which has been placed under tension beforehand by means of rollers or other means, or else placed in shape or smoothed out, an oxidizing composition also known as a “fixer”, so as finally to give the hair the desired shape.

This technique thus makes it possible either to make the hair wavy or to straighten it out or remove its curls, or alternatively to make it smooth.

The new shape given to the hair by a chemical treatment is long-lasting over time for a few weeks and especially withstands the action of washing with water or with shampoos, compared with techniques using styling products which lead to temporary reshaping, such as hair setting, this reshaping disappearing, however, on styling or washing with shampoo.

The reducing compositions generally used for the first step of a permanent-waving operation contain, as reducing agents, sulphites, bisulphites or, preferably, thiols.

In one of the methods for permanently reshaping the hair, which consists in simply placing the hair in shape without placing it under tension, in particular with thickened compositions, the rinsing operation with water after the reduction step leads to the destruction of the shape initially given, such that the hair, once treated with the fixing composition, does not have the shape initially desired. Moreover, the treated hair also has a tendency to lose its natural colour or the colour given to it by a colouring operation.

Patent FR-A-2 675 379 describes a process for permanently shaping the hair using a composition which has a predetermined viscosity and a predetermined bonding power, this process being carried out optionally without rinsing the hair after applying the reducing composition and before applying the oxidizing composition.

The process described in this document uses reducing compositions containing agents which give the composition considerable bonding properties of between 25 and 50 g, which is the force required to pull apart two standardized spatulas between which a precise amount of the reducing composition has been applied.

However, such compositions have the drawback of rinsing out poorly and of giving the treated and shaped hair poor cosmetic properties, especially on account of the sticky nature of the compositions used.

The Applicant has now discovered, and this forms the subject matter of the invention, a process and reducing compositions for shaping the hair which do not have the bonding properties defined in FR-A-2 675 379 and which give the hair, after the oxidizing composition has been applied, and without intermediate rinsing, permanent shapes which have the desired cosmetic properties.

The Applicant has moreover observed that the process leads to greater respect for the natural colour of the hair or for that of hair which has been coloured beforehand.

Finally, the process results in cost-effectiveness in terms of the compositions used.

A subject of the invention is thus a process for permanently reshaping the hair, comprising the application of a thickened reducing composition for shaping the hair and the application of a fixing or oxidizing composition without intermediate rinsing.

Other subjects of the invention will become apparent on reading the description and the examples which follow.

The process in accordance with the invention is a process for permanently shaping keratin fibres, and in particular the hair, this process being essentially characterized in that:

a keratin-reducing composition is applied to the keratin fibres, which have preferably been moistened, this composition preferably being thickened, having in particular a viscosity of between 0 and 2500 mPa·s, preferably between 100 and 2000 mPa·s and in particular between 200 and 1500 mPa·s at a laminar flow rate of 100 s⁻¹ and between 0 and 400 mPa·s at a laminar flow rate of 600 s⁻¹, this composition having a bonding power of less than 15 g, the reducing composition being applied to the fibres, which are preferably placed in shape without being placed under tension,

after an exposure time which is sufficient to reduce the keratin, an oxidizing composition is applied, preferably directly after the exposure time, without intermediate rinsing of the fibres,

after an exposure time which is sufficient to fix the fibres placed in shape and reduced during the first step, in a permanent shape, rinsing is carried out, preferably with water.

The viscosity is measured using a Rheostress RS 50 machine from Haake. The measurements are taken at 25° C. by using the removable spindle of reference number C35/2 sold with this machine and applying a laminar flow rate of 100 s⁻¹ or of 600 s⁻¹ for 60 seconds.

The hair is placed in shape by various means, such as rollers, clips, hair grips or simply by hand since a thickened composition is used.

The thickeners used in the reducing composition are preferably chosen from natural thickeners such as guar gum, tara gum or spruce meal.

These thickeners are used in the reducing composition in amounts that are sufficient to obtain the viscosities defined above.

The composition is said to be non-sticky if it does not pass the test described in FR-A-2 675 379 mentioned above.

The process for permanently shaping hair in accordance with the invention is preferably carried out by vaporizing the fixing composition on the hair.

Vaporization helps to avoid destroying the shaping carried out during the application of the thickened reducing composition. It also allows better control of the application of the fixing composition and more uniform impregnation of the hair.

The keratin-reducing composition contains a reducing agent chosen from sulphites, bisulphites and thiols.

Among the preferred compounds which may be mentioned are cysteine, cysteamine and their derivatives, such as their cosmetically acceptable salts, for instance the hydrochlorides, hydrobromides, citrates, acetates, sulphates, thiolactic acid, thioglycolic acid and their esters, in particular glyceryl thioglycolate. Thio-glycolic acid is particularly effective and constitutes the product most frequently used for reducing the disulphite linkages of keratin.

In one preferred embodiment, cysteine and its cosmetically acceptable salts will be used.

The reducing agents are present in proportions that are sufficient to reduce the —S—S— linkages, preferably between 1 and 25% and in particular between 1 and 10% by weight.

The pH of the reducing compositions is adjusted so as to have a pH of between 6.5 and 11.5.

The alkaline agents are preferably chosen from monoethanolamine, diethanolamine, triethanolamine, isopropylamine, 2-methyl-2-amino-1-propanol, propane-1,3-diamine, an ammonium or alkali metal carbonate or bicarbonate, aqueous ammonia, an organic carbonate such as guanidine carbonate, or an alkali metal hydroxide, which are used alone or as a mixture.

In addition to the thickeners mentioned above, the reducing composition can also contain other additives usually. used in these compositions and which do not interfere with the reducing properties of the composition.

The reducing composition can also contain nonionic, anionic, cationic or amphoteric surfactants commonly used in such compositions. Among these, mention may be made of alkyl sulphates, alkyl benzenesulphates, alkyl ether sulphates, alkyl sulphonates, quaternary ammonium salts, alkylbetaines, oxyethylenated alkyl-phenols, fatty acid alkanolamides, oxyethylenated fatty acid esters and nonionic surfactants of the hydroxypropyl ether family.

These surfactants are generally used in maximum proportions of 30%, and preferably between 0.5 and 10%, by weight relative to the total weight of the composition.

These compositions can also contain treating agents such as volatile or non-volatile, linear or cyclic silicones or mixtures thereof. Among the silicones which may be mentioned are polydimethylsiloxanes, quaternized polyorganosiloxanes as described in FR-A-2 535 730, polyorganosiloxanes containing an aminoalkyl group which are modified with alkoxycarbonylalkyl groups as described in patent U.S. Pat. No. 4,749,732, polyorganosiloxanes such as polydimethylsiloxane-polyoxyalkyl copolymers such as dimethicone copolyol, a polydimethylsiloxane containing stearoxy-(stearoxydimethicone) end groups, a poly-dimethylsiloxane dialkylammonium acetate copolymer or a polydimethylsiloxane polyalkylbetaine copolymer described in GB-A-2 197 352, polysiloxanes organomodified with mercapto or mercaptoalkyl groups as described in FR-B-1 530 369 and EP-A-0 295 780, as well as silanes such as stearoxytrimethylsilane.

Other ingredients which can be used in the reducing compositions in accordance with the invention are chosen from waxes, polymers chosen from cosmetically acceptable anionic, cationic, nonionic or amphoteric polymers, swelling agents and penetrating agents for reinforcing the efficacy of the reducing agent, such as dimethylisosorbitol, urea and its derivatives, pyrrolidone, n-alkylpyrrolidone, thiamorpholinone, alkyl ethers of alkylene glycol or of dialkylene glycol, such as, for example, propylene glycol monomethyl ether, dipropylene glycol monomethyl ether, C₃-C₆ alkanediols such as 1,2-propanediol, 2-imidazolidinone, as well as other compounds such as fatty alcohols, lanolin deriva-tives, ceramides, in particular ceramides themselves, the glycoceramides and the pseudoceramides described in particular in FR-A-95/12399, and in DOWNING Journal of Lipid Research, Vol. 35, p. 2060, 1994, or in FR-A-2 673 179, EP-A-0 227 994, WO-94/07844 or WO-92/05764, active ingredients such as pantothenic acid, agents for preventing hair loss, antidandruff agents, suspending agents, sequestering agents, opacifiers, colorants and sunscreens, as well as fragrances and preserving agents.

The oxidizing composition, preferably applied by vaporizing onto the hair which has been impregnated with the reducing composition and shaped, is an aqueous composition containing an oxidizing agent chosen from aqueous hydrogen peroxide solution, urea peroxides, bromates such as alkali metal bromates, persalts or a mixture of alkali metal bromates and a persalt.

According to one preferred embodiment, the oxidizing agent consists of aqueous hydrogen peroxide solution present in proportions of between 1 and 10 volumes and preferably of the order of 8 volumes.

When bromates are used, the concentration of alkali metal bromates is from 1 to 12% and that of persalts is from 0.1 to 15% by weight relative to the total weight of the oxidizing composition.

The pH of these compositions is generally acidic and it is usually between 2 and 9 and preferably between 3 and 8.

When aqueous hydrogen peroxide solution is used, it can be stabilized with phenacetin, acetaniline, mono- and trisodium phosphates or with 8-hydroxyquinoline sulphates.

Application of the oxidizing composition is carried out, preferably by vaporizing, after an exposure time which is sufficient to reduce the —S—S— linkages, generally of between 2 and 30 minutes and preferably of between 5 and 20 minutes. A bonnet or protective gel can be used during this waiting period.

The oxidizing composition is kept in contact with the hair for a period which is sufficient to fix the permanent reshaping, this period being from about 5 to 30 minutes and in particular between 5 and 15 minutes, followed by thorough rinsing with water of the hair thus treated, drying and styling for the permanent shaping.

The examples which follow are intended to illustrate the invention without, however, being limited in nature.

### EXAMPLE

### Example 1

| Reducing agent | | |
|---|---|---|
| Thioglycolic acid | 5 | g |
| Diethylenetriaminepentaacetic acid, | 0.2 | g |
| pentasodium salt, as an aqueous 40% | | |
| solution | | |
| Aqueous ammonia containing 20% ammonia | qs pH 7.9 | |
| Ammonium carbonate | 5 | g |
| Fragrance | 0.5 | g |
| Oxyethylenated (20 mol of ethylene | 1 | g |
| oxide) oleyl alcohol | | |
| Demineralized water | qs 100 | g |
| Fixing agent | | |
| Aqueous hydrogen peroxide | 4.8 | g |
| solution containing 50% | | |
| hydrogen peroxide | | |
| Stabilizers | 0.2 | g |
| Citric acid | qs pH 3 | |
| Demineralized water | qs 100 | g |

The non-sticky liquid reducing agent is applied to the washed and drip-dry hair. The hair is placed in shape, for example by forming curls held with clips. The hair is left to stand for 15 minutes and the fixing agent is then vaporized onto it uniformly. The hair is left to stand for 5 minutes. The hair is then rinsed.

Long-lasting reshaping of the fibre is thus obtained simply and quickly, thus allowing easier restyling over time.

### Example 2

| Reducing agent | | |
|---|---|---|
| Cysteine | 3 | g |
| Guar gum | 1 | g |
| Spruce meal | 8 | g |
| Monoethanolamine | 2.2 | g |
| Fragrance | 0.5 | g |
| Oxyethylenated (20 mol of ethylene | 1 | g |
| oxide) oleyl alcohol | | |
| Cocoylamidopropylbetaine/glyceryl | 1.8 | g |
| monolaurate mixture as an aqueous | | |
| 30% solution | | |
| Diethylenetriaminepentaacetic acid, | 0.2 | g |
| pentasodium salt, as an aqueous 40% | | |
| solution | | |
| Dimethyldiallylammonium chloride | 2.5 | g |
| homopolymer as an aqueous 40% | | |
| solution | | |
| Demineralized water | qs 100 | g |
| Fixing agent | | |
| Sodium bromate | 7.5 | g |
| Phosphate buffer | pH 7.5 | |
| Demineralized water | qs 100 | g |

The lightly thickened reducing agent (1400 mPa·s at 100 s⁻¹ and 200 mPa·s at 600 s⁻¹), with a bonding power equal to 10 mg, of pH 9.1, allows the hair to be placed in shape in the desired position without the aid of equipment for placing it under tension.

This product is applied to the washed and drip-dry hair, after which the hair is placed in shape. The hair is left to stand for 15 minutes, after which the fixing agent is vaporized on it. The hair is left to stand for 10 minutes and is then rinsed. The products are easy to remove by rinsing.

Long-lasting reshaping of the hair is obtained, thus allowing easier styling over time.

1. Process for permanently shaping keratin fibres, comprising:
applying to the keratin fibres a keratin-reducing composition which has a bonding power of less than 15 g and a viscosity of between 100 and 2000 mPa·s at a laminar flow rate of 100 s⁻¹, and shaping said fibres,
applying an oxidizing composition to said fibres after said fibres have been reduced, without intermediate rinsing of the fibres, and
rinsing said fibres after an exposure time which is sufficient to fix the fibers placed in shape and reduced during the first step in a permanent shape,
the reducing composition being thickened with natural thickeners selected from the group consisting of quar gum, tara gum and spruce meal.

2. Process according to claim 1, wherein the oxidizing composition is applied directly after said fibres have been reduced.

3. (canceled)

4. Process according to claim 1 wherein the viscosity is between 200 and 1500 mPa·s at a laminar flow rate of 100 s⁻¹.

5-6. (canceled)

7. Process according to claim 1 wherein said reducing composition has a bonding power of less than 15 g, corresponding to the force required to pull apart 2 standardized spatulas between which a determined amount of the reducing composition has been applied.

8. Process according to claim 1 wherein the oxidizing composition is applied to the fibres, placed in shape with the thickened reducing composition, by vaporization.

9. Process according to claim 1 wherein the keratin-reducing composition contains a reducing agent selected from the group consisting of sulphites, bisulphites and thiols.

10. Process according to claim 1 wherein the keratin-reducing agent is selected from the group consisting of cysteine, cysteamine, a cosmetically acceptable salt of cysteine, a cosmetically acceptable salt of cysteamine, thiolactic acid, thioglycolic acid, a cosmetically acceptable ester of thiolactic acid and a cosmetically acceptable ester of thioglycolic acid.

11. Process according to claim 1 wherein the keratin-reducing agent is present at a concentration of between 1 and 25% by weight.

12. Process according to claim 1 wherein the reducing composition has a pH of between 6.5 and 11.5.

13. Process according to claim 12 wherein the pH is adjusted using absorbed agents selected from the group consisting of monoethanolamine, diethanolamine, triethanolamine, isopropylamine, 2-methyl-2-amino-1-propanol, propane-1,3-diamine, an ammonium or alkali metal carbonate or bicarbonate, aqueous ammonia, an organic carbonate and an alkali metal hydroxide, which are used alone or as a mixture.

14. Process according to claim 1 wherein the reducing composition further contains nonionic, anionic, cationic or amphoteric surfactants.

15. Process according to claim 1 wherein the reducing composition contains treating agents chosen from volatile or non-volatile, linear or cyclic silicones or mixtures thereof, waxes, polymers, penetrating agents, swelling agents, alkyl ethers of alkylene glycol or of dialkylene glycol, alkanediols, fatty alcohols, lanolin derivatives, ceramides, active ingredients, agents for preventing hair loss, antidandruff agents, suspending agents, sequestering agents, opacifiers, colorants, sunscreens, fragrances and preserving agents, or mixtures thereof.

16. Process according to claim 1 wherein the oxidizing composition is applied by vaporizing said composition, directly and without intermediate rinsing, onto the keratin fibres which have been placed in shape and reduced by the reducing composition, after an exposure time of the reducing composition of between 2 and 30 minutes.

17. Process according to claim 1 wherein the oxidizing agent in the fixing composition is selected from the group consisting of hydrogen peroxide, urea peroxide, bromates, persalts and mixtures thereof.

18. Process according to claim 1 wherein the oxidizing composition is kept in contact with the fibres for a period of from 1 to 30 minutes.
